(19) United States
(12) Patent Application Publication
(10) Pub. No.: US 2021/0307723 A1
(43) Pub. Date: Oct. 7, 2021

(54) SPATIAL REGISTRATION METHOD FOR IMAGING DEVICES

(71) Applicant: Trig Medical Ltd., Nesher (IL)

(72) Inventors: Yoav Paltieli, Haifa (IL); Ishay Perez, Nesher (IL)

(73) Assignee: Trig Medical Ltd., Nesher (IL)

(21) Appl. No.: 16/766,726

(22) PCT Filed: Nov. 13, 2019
(86) PCT No.: PCT/IB2019/059755

§ 371 Date: May 25, 2020

Related U.S. Application Data
(60) Provisional application No. 62/768,929, filed on Nov. 18, 2018.

(51) Int. Cl. A61B 8/00; A61B 8/12; G06T 7/30

(52) U.S. Cl. CPC A61B 8/4254; A61B 8/40; G06T 2207/30004; G06T 7/30; G06T 2207/10132; A61B 8/12

(57) ABSTRACT

A method is provided for registration of images obtained of a patient in real time with respect to a tracking device. The method is not based on internal or external fiducial markers attached to the patient, but rather the registration is done relative to a reference plate of a guiding system affixed to a table that supports the patient.

### FIELD OF THE INVENTION

The present invention relates generally to registration of the location and orientation of a sensor with respect to the image plane of an imaging transducer.

### BACKGROUND OF THE INVENTION

There are medical systems that are used for guiding instruments by means of position sensors and imaging probes. For example, the absolute location and orientation of the plane displayed by the imaging system (the image plane) may be determined by means of a position sensor placed on the imaging probe. If it is desired to track the path and position of a needle, for example, the tracking system must be able to track the position of the needle relative to the images acquired by the imaging system.

One way of tracking the needle is to affix a needle position sensor to a predetermined point on the needle, and measure the precise location and orientation of the needle tip. However, the imaging position sensor, which is attached to the imaging transducer at a convenient, arbitrary location thereon, does not have a well-determined spatial position and orientation to the image plane of the transducer so as to precisely relate the transducer position sensor to the transducer imaging plane. Since the navigation of the needle to the anatomical target uses the acquired images as a background for the display of the needle and its future path, it is imperative to calculate the precise location and orientation of the imaging plane with respect to the position sensor on the imaging transducer.

Fusion imaging is a technique that fuses two different imaging modalities. For example, in certain medical procedures, such as but not limited to, hepatic intervention, real-time ultrasound is fused with other imaging modalities, such as but not limited to, CT, MR, and positron emission tomography PET-CT and others. Fusing imaging requires registration of the ultrasonic images with the other imaging modality images. Prior art imaging registration requires registering images relative to fiducial markers (either internal or external to the patient).

### SUMMARY OF THE INVENTION

The present invention seeks to provide improved methods for registration of the position and orientation of the position sensor mounted on the imaging probe (which may be, without limitation, an ultrasonic probe), as is described more in detail hereinbelow. The terms “probe” and “transducer” are used interchangeably throughout.

The position sensor, also referred to as a tracking device, may be, without limitation, magnetic, optical, electromagnetic, RF (radio frequency), IMU (inertial measurement unit), accelerometer and/or any combination thereof.

The tracking device is fixed on the imaging transducer, thereby defining a constant spatial relation that is maintained between the position and orientation of the tracking device and the position and orientation of the image plane of the imaging transducer.

Calibration methods may be used to find this constant spatial relation. One non-limiting suitable calibration method is that of U.S. Pat. No. 8,887,551, assigned to Trig Medical Ltd., Israel, the disclosure of which is incorporated herein by reference. By using this constant spatial relation, a processor can calculate the exact position and orientation of the image based on the position and orientation of the tracking device.

In order to use such a calibration method, a registration procedure must be performed in order to register the image (e.g., ultrasonic image) with respect to the attached tracking device.

The present invention provides a method for performing this registration procedure using images of the imaging device (e.g., pictures of the ultrasound transducer) together with the attached tracking device using image processing techniques, as is described below.

This method requires the use of an imaging device (e.g., camera, X-Ray, CT) to take one or more images of the image transducer from one or more angles or to capture a video-clip in which the image transducer is viewed continuously from one or more angles. The tracking device appears in one or more of the acquired images. The tracking device shape and size must be known.

There is thus provided in accordance with an embodiment of the present invention a method for registration of images with respect to a tracking device including acquiring an image of an imaging transducer to which is attached a tracking device, identifying shapes and dimensions of the imaging transducer and the tracking device, calculating spatial orientations of the imaging transducer and the tracking device, calculating transformation matrix based on the spatial orientations of the imaging transducer and the tracking device, transforming imaging transducer coordinates to attached tracking device coordinates, thereby providing registration of the image with the imaging transducer, calculating an image plane of the imaging transducer, and assuming the image plane is in a constant and well-known spatial relation to the transducer body.

The image of the imaging transducer may include a portion of the imaging transducer that emits imaging energy, the tracking device, and a fiducial marker of the imaging transducer. The identifying step may include finding an outline of the imaging transducer and the portion that emits the imaging energy, the tracking device and the fiducial marker.

The step of calculating of the spatial orientation may include calculating a distance between any points of interest in the image using the tracking device shape as a reference.

The step of determining of the spatial position of the image plane may include determining a spatial location of each pixel of the image.

The method may further include affixing a position sensor to an invasive instrument to obtain positional data of the invasive instrument during an invasive procedure, and using the tracking device to register the positional data with respect to the image plane of the imaging transducer.

### BRIEF DESCRIPTION OF THE DRAWINGS

The present invention will be understood and appreciated more fully from the following detailed description, taken in conjunction with the drawings in which:

FIG. 1 is a simplified pictorial illustration of a position sensor (tracking device) mounted on an imaging probe (transducer), in accordance with a non-limiting embodiment of the present invention, and showing the image plane of the probe;

FIG. 2 is a simplified block diagram of a method for registration of images with respect to a tracking device, in accordance with a non-limiting embodiment of the present invention; and

FIGS. 3A and 3B are simplified illustrations of a reference plate, imaging table and position sensor, in accordance with a non-limiting embodiment of the present invention.

### DETAILED DESCRIPTION OF EMBODIMENTS

Reference is now made to FIG. 1, which illustrates a position sensor (tracking device) 10 mounted on an imaging probe (transducer) 12. FIG. 1 shows the image plane of the probe 12. The probe 12 has a fiducial mark 14, such as a lug or protrusion on the left and/or right side of probe 12.

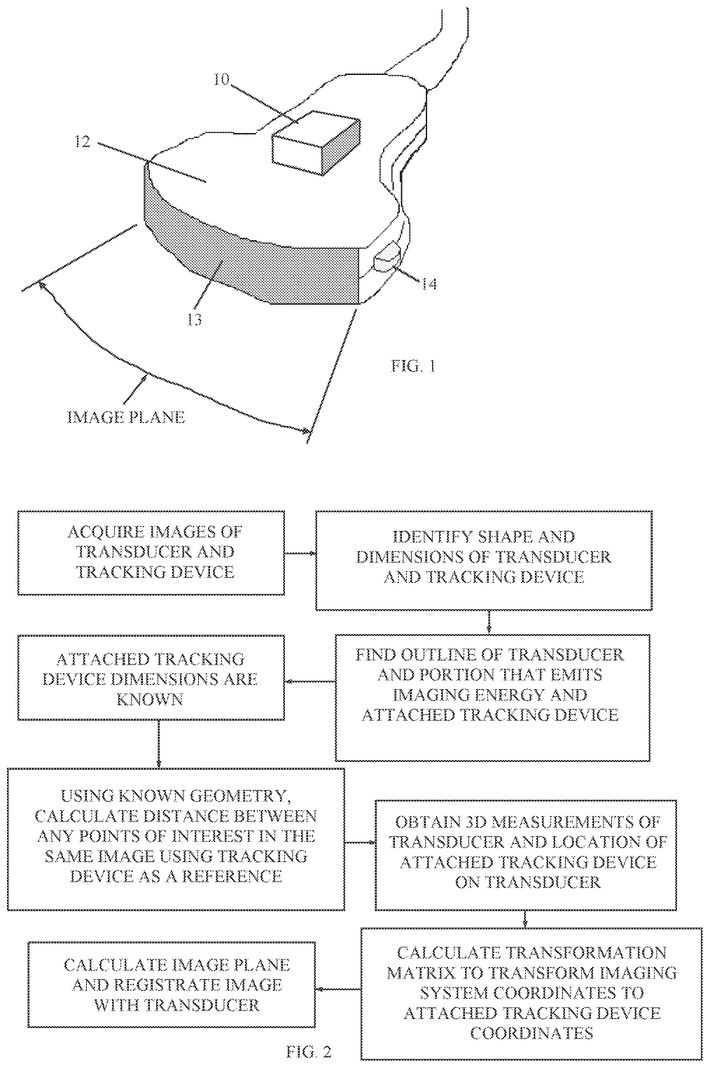

The following is one non-limiting description of a method of the invention and the description follows with reference to FIG. 2.

Step 1—Acquisition of Pictures/Video Clip (the Term “Image” Encompasses Pictures, Photos, Video Clips and the Like).

One or more images of the transducer with the attached tracking device are acquired. In the acquired images the following are visible:

a. The transducer including the portion of the transducer that emits the ultrasonic energy (or other imaging modality energy, such as RF).

b. The attached tracking device.

c. The fiducial marker of the transducer, such as a left or right side notch or marker on the transducer.

Step 2—Identification of Shapes and Dimensions Using Image Processing Techniques

After acquiring the images, image processing techniques, well known in the art and commercially available, are used to identify the shape of the transducer and the attached tracking device. This identification process finds the outline of the transducer and the portion 13 (FIG. 1) that emits the imaging energy (e.g., ultrasonic waves), the attached tracking device and the fiducial marker.

Step 3—Calculation of the 3D Dimensions and Spatial Orientations of the Identified Items

The attached tracking device dimensions are known. Using this known geometry, the processor calculates the distance between any points of interest in the same picture (image) using the tracking device geometry as a reference. After the outline and details of the transducer and attached tracking device are identified in one or more images, the identified items are analyzed in order to obtain 3D position and orientation of the portion that emits the imaging energy 13 and the fiducial marker 14, in reference to the tracking device.

Step 4—Calculation of the Transformation Matrix

Based on the measurements and relative location and orientation of the attached tracking device relative to the transducer, the transformation matrix is calculated, which will be used to transform the imaging system coordinates to the attached tracking device coordinates. This matrix represents the registration of the image (e.g., ultrasonic image) with the transducer.

Step 5—Calculation of the Image Plane

Since the image plane is in a constant and well-known position relative to the transducer, the spatial position of the image plane relative to the tracking device is determined. Furthermore, using scales presented on the image, the spatial location of each pixel of the image relative to the tracking device is determined.

Some of the applicable positioning systems and tracking devices for use with the registration procedure of the invention include, but are not limited to:

a. A magnetic positioning system where the tracking device is a magnet or magnetic sensor of any type or a magnetic field source generator.

b. An electromagnetic positioning system where the tracking device is an electromagnetic sensor of any type or an electromagnetic source generator.

c. An ultrasonic positioning system where the tracking device is an ultrasonic sensor (or microphone) of any type or an ultrasonic source generator (transmitter or transducer).

d. An optical positioning system where the tracking device is used as allocation/orientation marker or a light source of any type.

e. A positional system and device other than the above systems or a system that is constructed as any combinations of the above systems.

The spatial position and orientation of the instrument to be tracked, e.g., a needle, is overlaid on the ultrasonic image in real time allowing planning before insertion and showing the expected position and orientation of the needle during the insertion in both in-plane and out-of-plane procedures.

Further features include taking into account the examination (imaging) table used for the patient and the invasive instrument guiding system. The position of the examination (imaging) table with respect to the image plane (CT, MRI, X-ray, etc.) is known and documented on the image. This relative position can be obtained via the DICOM (Digital Imaging and Communications in Medicine) protocols.

Interventional procedures under CT, MR, and X-ray imaging require registration of the scanned images. Prior art imaging registration requires registering images relative to internal or external fiducial markers attached to the patient. In contrast, the present invention provides a novel registration technique which is not based on internal or external fiducial markers attached to the patient, but rather the registration is done relative to a base plate (reference plate) 50 that includes position sensors or transmitters of any type, such as but not limited to, optical, ultrasonic, RF, electromagnetic, magnetic, IMU and others.

It is assumed that the invasive instrument guiding system has a reference plate. In order to know the position of the invasive instrument guiding system, one can place the invasive instrument guiding system on the examination table so that the reference plate is fixed to the table, and obtain an image of the plate on the examination table. The system identifies the plate (or known structure fixed to the plate) relative to the position of the imaging table according to the table structure or fiducial mark on the table.

The 3D coordinates of the reference plate 50 are known and defined with respect to a known structure 54 of the other imaging modality, such as the imaging table. The location of the imaging table is defined in each imaging slice. The 3D coordinates of the reference plate 50 may then be defined with respect to the imaging table (known structure 54).

At least one sensor can be affixed to the patient to compensate for any movements of the patient relative to the reference plate and the imaging table during imaging. The assumption is that the plate does not move until after performing the scan (from obtaining an image of the plate on the examination table until scanning of the patient by CT, MRI, X-ray, etc.).

After scanning, the positions of the scanning slices are registered relative to the plate 50, whose position relative to the scanning table is known. Thus, the plate can be in any arbitrary position, since the position of the patient is established relative to the plate during scanning.

A position sensor is affixed to the invasive instrument (e.g., needle) to obtain positional data of the invasive instrument during the invasive procedure.

The spatial position and orientation of the insertion tool (e.g. needle) is overlaid in real time on the CT/MR/PETCT/X-ray sagittal image which includes the target, allowing planning before insertion and showing the expected position and orientation of the needle during the insertion in both—in-plane and out-of-plane procedures.

Another option is to use known algorithms of multi-planar reconstruction (MPR), which provide efficient computation of images of the scanned volume that can create multi-planar displays in real-time. The spatial position of any section of the MPR volume and slices in relation to the plate is calculated based on the known spatial position of the previously scanned sagittal image sections. The system presents in real time one or more cross-sections of the registered volume passing through the needle allowing out-of-plane procedure at any needle angle, with the advantage of showing the complete needle in the rendered images (as in-plane procedures).

Another option is to use at least one image slice displaying the image of an external or internal feature of the plate with a particular geometry (e.g., pyramid, polyhedron and the like) as the reference for the plate position with respect to that slice(s). Since the spatial relationship of all slices in the scanning volume is known, the spatial position of the plate in relation to all image slices is determined.

The imaging system obtains images of the position sensor that is affixed to the needle (or other invasive instrument) and two other points on the invasive instrument. The two points may be chosen so that the length of the invasive instrument can be calculated by the imaging processor (the invasive instrument length can alternatively be entered by hand).

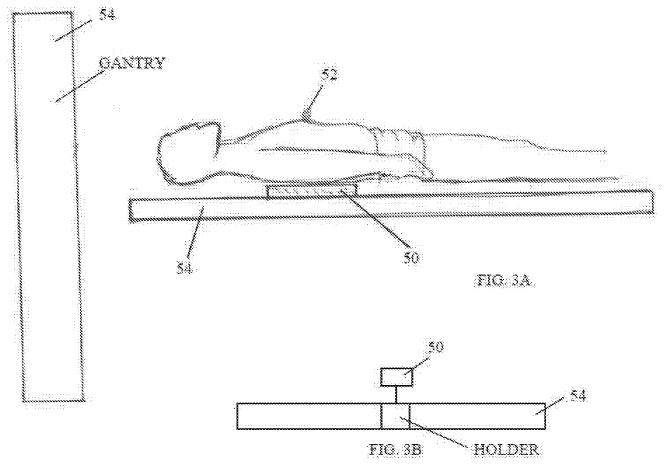

Reference is made to FIGS. 3A and 3B, which illustrate a reference plate, imaging table and position sensor, in accordance with a non-limiting embodiment of the present invention.

As mentioned above, fusion imaging requires registration of the ultrasonic images with the other imaging modality images. Prior art imaging registration requires registering images relative to fiducial markers (either internal or external to the patient). In contrast, the present invention provides a novel registration technique which is not based on internal or external fiducial markers, but rather the registration is done relative to a base plate (reference plate) 50 that includes position sensors or transmitters of any type, such as but not limited to, optical, ultrasonic, RF, electromagnetic, magnetic, IMU and others.

In an embodiment of the invention, the position of the patient relative to the plate 50 is established by affixing a position sensor to the patient. The position of the patient as sensed by the position sensor when obtaining the image slice of the target in the patient serves as the basis for calculating the position of the patient during an invasive procedure such as needle insertion. The position sensor does not move, such as being placed in bone, instead of soft tissues that can move. However, if the position sensor does move, this movement can be sensed and taken into account by using it and/or other position sensors, e.g., mounted on the skin over the ribs or under the diaphragm to cancel the effects of breathing or other factors. The information from the position sensor(s) that detect breathing effects may be used to instruct the patient when to hold his/her breath during the invasive procedure or during fusion of images. This information can also be used to indicate in real-time the degree of similarity between the patient current breathing state and the one in the slice being displayed.

What is claimed is:

1. A method for registration of images obtained of a patient in real time with respect to a tracking device, the method comprising:
supporting a patient on a table, wherein a reference plate of a guiding system is spatially fixed with respect to said table;
tracking an object related to the patient with a tracking device of said guiding system;
obtaining images of said object in real time with an imaging system, wherein said table is defined in each of said images; and
disregarding any internal markers in the patient and disregarding any external fiducial markers attached to the patient, and instead registering said images obtained in real time with said tracking device with respect to said reference plate.

2. The method according to claim 1, wherein said reference plate comprises a reference plate position sensor or a reference plate transmitter.

3. The method according to claim 1, further comprising affixing at least one compensating position sensor to the patient to compensate for any movements of the patient relative to the table during imaging.

4. The method according to claim 1, wherein a spatial position and orientation of an object is overlaid in real time on said image which includes a target of interest.

5. The method according to claim 4, wherein the object comprises an insertion tool.

6. The method according to claim 1, wherein said tracking device is part of a magnetic positioning system.

7. The method according to claim 1, wherein said tracking device is part of an electromagnetic positioning system.

8. The method according to claim 1, wherein said tracking device is part of an ultrasonic positioning system.

9. The method according to claim 1, wherein said tracking device is part of an optical positioning system.

10. A method for registration of images with respect to a tracking device comprising:
acquiring an image of an imaging transducer to which is attached a tracking device;
identifying shapes and dimensions of said imaging transducer and said tracking device;
calculating spatial orientations of said imaging transducer and said tracking device;
calculating a transformation matrix based on the spatial orientations of said imaging transducer and said tracking device;
using said transformation matrix to transform imaging system coordinates to attached tracking device coordinates, thereby providing registration of said image with said imaging transducer;
calculating an image plane of said imaging transducer relative to the tracking device; and
assuming said image plane is in a constant and well-known position relative to said imaging transducer, determining a spatial position of said image plane.

11. The method according to claim 10, wherein the image of said imaging transducer includes a portion of said imaging transducer that emits imaging energy, said tracking device, and a fiducial marker of said imaging transducer.

12. The method according to claim 11, wherein the identifying comprises finding an outline of said imaging transducer and said portion that emits the imaging energy, said tracking device and said fiducial marker.

13. The method according to claim 10, wherein the calculating of the spatial orientations comprises calculating a distance between any points of interest in said image using said tracking device as a reference.

14. The method according to claim 10, wherein the determining of the spatial position of said image plane comprises determining a spatial location of each pixel of said image.

15. The method according to claim 10, comprising affixing a position sensor to an invasive instrument to obtain positional data of said invasive instrument during an invasive procedure, and using said tracking device to register said positional data with respect to said image plane of said imaging transducer.
